(19) United States
(12) Patent Application Publication
(10) Pub. No.: US 2026/0060569 A1
(43) Pub. Date: Mar. 5, 2026

(54) INFORMATION PROCESSING APPARATUS, INFORMATION PROCESSING METHOD, INFORMATION PROCESSING PROGRAM, AND TRAINED MODEL

(71) Applicant: FUJIFILM Corporation, Tokyo (JP)

(72) Inventor: Takeshi HIRAYU, Tokyo (JP)

(73) Assignee: FUJIFILM Corporation, Tokyo (JP)

(21) Appl. No.: 19/313,887

(22) Filed: Aug. 29, 2025

(30) Foreign Application Priority Data
Sep. 5, 2024 (JP) 2024-153481

(51) Int. Cl. A61B 5/103; G06T 7/90; G06V 10/56; G16H 50/20; G16H 50/30

(52) U.S. Cl. CPC A61B 5/1032; G06T 7/90; G06V 10/56; G16H 50/20; G16H 50/30; G06T 2207/10024

(57) ABSTRACT

An information processing apparatus including a processor, wherein the processor is configured to: acquire color information of at least a part of a body surface of a subject undergoing imaging for image diagnosis; and monitor a health state of the subject undergoing the imaging using an estimation model for estimating a change in the color information in a case where the health state of the subject changes.

### CROSS-REFERENCE TO RELATED APPLICATIONS

This application claims priority from Japanese Application No. 2024-153481, filed on Sep. 5, 2024, the entire disclosure of which is incorporated herein by reference.

### BACKGROUND

### Technical Field

The present disclosure relates to an information processing apparatus, an information processing method, an information processing program, and a trained model.

### Related Art

In the related art, various technologies for grasping a health state of a user are known.

For example, JP2022-188611A discloses a technology of recognizing a facial expression of a patient based on continuously recorded face information of the patient, generating an indicator indicating a time-series change in the facial expression using the face information and medical data of the patient, and displaying the indicator and the facial expression together with a time-series change in the medical data. In addition, for example, JP2018-183509A discloses a technology of recognizing a predetermined user from an analysis target image generated by imaging a front side of a mirror, and analyzing a physical state based on a determination criterion feature amount of the user and an analysis time feature amount obtained from the analysis target image.

In recent years, with advancements in medical devices such as a computed tomography (CT) apparatus and a magnetic resonance imaging (MRI) apparatus, high-resolution three-dimensional images with higher quality have been used for image diagnosis. In image diagnosis using the CT apparatus, and the MRI apparatus, a contrast agent may be used for more detailed diagnosis, but the subject may be in poor physical condition due to the influence of the contrast agent. In addition, even in a case where the contrast agent is not used, the condition of the subject may change rapidly, or the subject may be in poor physical condition due to a nervous state. Therefore, there is a demand for a technology that can monitor the health state of the subject undergoing imaging for image diagnosis.

### SUMMARY

The present disclosure provides an information processing apparatus, an information processing method, an information processing program, and a trained model that can monitor a health state of a subject undergoing imaging for image diagnosis.

A first aspect of the present disclosure is an information processing apparatus comprising: a processor, in which the processor is configured: to acquire color information of at least a part of a body surface of a subject undergoing imaging for image diagnosis; and monitor a health state of the subject undergoing the imaging using an estimation model for estimating a change in the color information in a case where the health state of the subject changes.

The estimation model may be a learning model that is trained in advance to receive input of reference color information that is the color information serving as a reference for the health state of the subject and output abnormal color information that is the color information in a case where the health state of the subject is abnormal. The processor may be configured to: acquire, as the reference color information, the color information of the body surface of the subject before a start of the imaging; generate the abnormal color information by inputting the reference color information to the estimation model; and estimate that the health state of the subject is abnormal in a case where a comparison result between the color information during the imaging and the abnormal color information satisfies a predetermined condition.

The estimation model may output a plurality of pieces of the abnormal color information in a case where the health state of the subject indicates different types of abnormal states. The processor may be configured to estimate that the health state of the subject is abnormal in a case where a comparison result between the color information during the imaging and at least one of the plurality of pieces of abnormal color information satisfies a predetermined condition.

The estimation model may be trained by machine learning using a combination of the color information in a case where the health state is normal and the color information in a case where the health state is abnormal as training data.

The processor may be configured to issue a warning in a case where it is estimated that the health state of the subject is abnormal.

The processor may be configured to extract the color information from a visible light image obtained by performing visible light imaging on the subject.

The processor may be configured to extract the color information in a predetermined region of the body surface of the subject from the visible light image.

The color information may be color information in at least one of a skin or a mucous membrane of the subject.

The color information may be color information in a face of the subject.

The imaging may be imaging accompanied by administration of a contrast agent.

A plurality of the estimation models may be generated according to the color information in a case where the health state of the subject is normal.

A second aspect of the present disclosure is an information processing method executed by a computer, comprising: acquiring color information of at least a part of a body surface of a subject undergoing imaging for image diagnosis; and monitoring a health state of the subject undergoing the imaging using an estimation model for estimating a change in the color information in a case where the health state of the subject changes.

A third aspect of the present disclosure is an information processing program causing a computer to execute a process comprising: acquiring color information of at least a part of a body surface of a subject undergoing imaging for image diagnosis; and monitoring a health state of the subject undergoing the imaging using an estimation model for estimating a change in the color information in a case where the health state of the subject changes.

A fourth aspect of the present disclosure is a trained model for causing a computer to function as follows: receiving input of reference color information that is color information of at least a part of a body surface of a subject on whom imaging for image diagnosis is performed and serves as a reference for a health state of the subject; and outputting abnormal color information that is the color information in a case where the health state of the subject is abnormal, in which the trained model is trained in advance by machine learning using a combination of the color information in a case where the health state is normal and the color information in a case where the health state is abnormal as training data.

According to the above-described aspect, the information processing apparatus, the information processing method, the information processing program, and the trained model of the present disclosure can monitor the health state of the subject undergoing imaging for image diagnosis.

### BRIEF DESCRIPTION OF THE DRAWINGS

FIG. 1 is a perspective view showing an outline of a CT apparatus.

FIG. 2 is a block diagram showing an example of a hardware configuration of an information processing apparatus.

FIG. 3 is a block diagram showing an example of a functional configuration of the information processing apparatus.

FIG. 4 is a diagram for describing processing of the information processing apparatus.

FIG. 5 is a diagram for describing processing of the information processing apparatus.

FIG. 6 is a diagram showing an example of a screen displayed on a display.

FIG. 7 is a flowchart showing an example of information processing.

### DETAILED DESCRIPTION

Hereinafter, an example of embodiments of the disclosed technology will be described with reference to the drawings. The same or equivalent components and parts in the respective drawings are denoted by the same reference numerals, and the duplicated description will be omitted. In addition, dimensional ratios in the drawings are exaggerated for convenience of description, and may be different from the actual ratios.

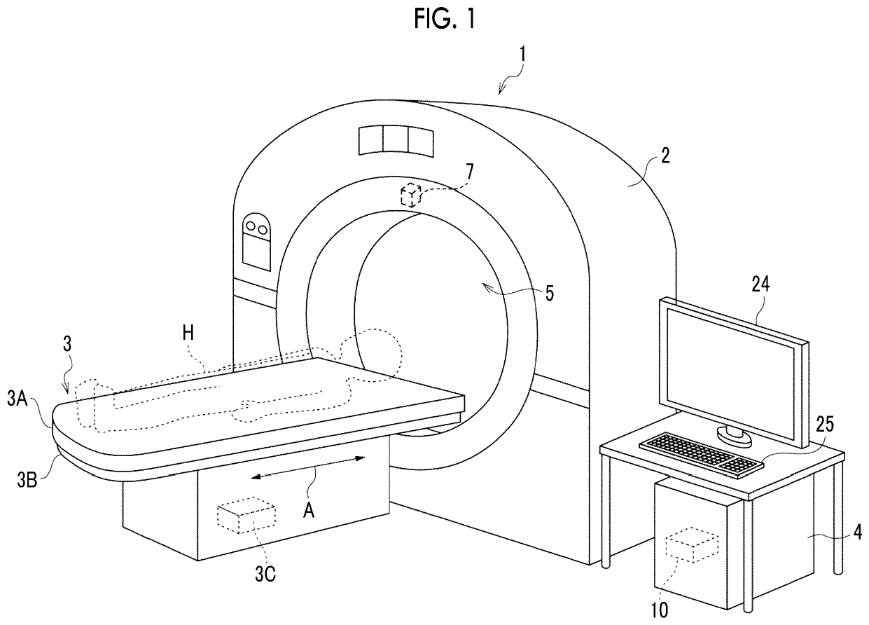

A configuration of a computed tomography (CT) apparatus 1 will be described with reference to FIG. 1. As shown in FIG. 1, the CT apparatus 1 according to the present embodiment comprises a gantry 2, an examination table 3, and a console 4. The gantry 2, the examination table 3, and the console 4 are configured to be connectable to each other via a wired or wireless network (not shown). The console 4 may be disposed in a remote state (for example, a separate room) from the gantry 2 and the examination table 3.

The gantry 2 has a tunnel-shaped structure with an opening portion 5 at the center thereof. A radiation source unit that emits X-rays and a detection unit that detects the X-rays to generate a radiation image are provided inside the gantry 2 (both not shown). The radiation source unit and the detection unit can be each rotated along an annular shape of the gantry 2 in a state of maintaining a positional relationship in which the radiation source unit and the detection unit face each other. Further, a controller that controls an operation of the CT apparatus 1 is provided inside the gantry 2.

In addition, the gantry 2 includes a camera 7 that performs visible light imaging on a subject H undergoing imaging for image diagnosis. The camera 7 is a camera that can capture a color image of red (R), green (G), and blue (B) by detecting reflected light of the subject H. The camera 7 includes an imaging element such as a lens and a charge coupled device (CCD), and acquires a visible light image, which is a moving image, by imaging the subject H on the examination table 3 at a predetermined frame rate and outputs the visible light image to the console 4.

Specifically, the camera 7 may be provided near the radiation source unit so as to be able to image the face of the subject H transported into the opening portion 5 of the gantry 2 (see FIG. 1). In addition, the camera 7 may be provided on a ceiling or a wall surface of the imaging room, the housing of the gantry 2, or the like so as to image the face of the subject H in a state where the face is exposed from the opening portion 5. In addition, the camera 7 may be provided at each of the plurality of locations as described above.

The subject H is placed on the examination table 3. The examination table 3 comprises an examination table portion 3A on which the subject H lies down, a base portion 3B that supports the examination table portion 3A, and a driving unit 3C that reciprocates the examination table portion 3A in an arrow A direction. The examination table portion 3A can be slid with respect to the base portion 3B in the arrow A direction via the driving unit 3C. In a case where the CT image is captured, the examination table portion 3A is slid, and the subject H lying down on the examination table portion 3A is transported into the opening portion 5 of the gantry 2.

In the CT apparatus 1, the CT image is captured by driving the gantry 2 and the examination table 3 in response to an input of an operator such as a technician of the console 4. The console 4 includes the information processing apparatus of the present disclosure. The information processing apparatus according to the present embodiment has a function of monitoring a health state of the subject H undergoing the imaging for the image diagnosis. An example of a configuration of the information processing apparatus 10 according to the present embodiment will be described below.

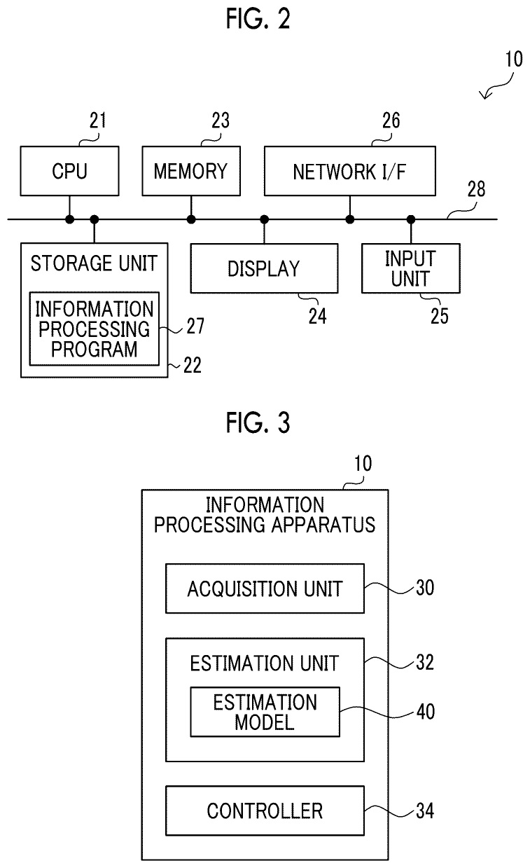

First, an example of a hardware configuration of the information processing apparatus 10 according to the present embodiment will be described with reference to FIG. 2. The information processing apparatus 10 includes a central processing unit (CPU) 21, a non-volatile storage unit 22, and a memory 23 as a temporary storage area. Further, the information processing apparatus 10 includes a display 24, an input unit 25, and a network interface (I/F) 26. The CPU 21, the storage unit 22, the memory 23, the display 24, the input unit 25, and the network I/F 26 are connected to each other via a bus 28, such as a system bus and a control bus, so that various types of information can be exchanged.

The storage unit 22 is implemented by a storage medium such as, for example, a hard disk drive (HDD), a solid state drive (SSD), and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor according to the present disclosure.

The display 24 is, for example, a liquid crystal display, and displays various types of information. The input unit 25 includes a pointing device such as a mouse and a keyboard, and is used to perform various types of input to the device. The display 24 may be configured by a touch panel and may also be used as the input unit 25. The display 24 and the input unit 25 are also shown in FIG. 1.

The network I/F 26 is an interface for communicating with various devices including the CT apparatus 1 via a network. For the communication, for example, a wired communication standard, such as Ethernet (registered trademark) or fiber distributed data interface (FDDI), or a wireless communication standard, such as 4G, 5G, or Wi-Fi (registered trademark), is used. As the information processing apparatus 10, for example, a server computer, a personal computer, a smartphone, a tablet terminal, a wearable terminal, or the like can be applied as appropriate.

Next, an example of a functional configuration of the information processing apparatus 10 according to the present embodiment will be described with reference to FIG. 3. As shown in FIG. 3, the information processing apparatus 10 includes an acquisition unit 30, an estimation unit 32, and a controller 34. The CPU 21 functions as the acquisition unit 30, the estimation unit 32, and the controller 34 by executing the information processing program 27.

The acquisition unit 30 acquires color information of at least a part of a body surface of the subject H undergoing the imaging for the image diagnosis. The imaging for the image diagnosis is not the visible light imaging by the camera 7, but means, for example, the CT imaging by the CT apparatus 1, and hereinafter, is referred to as “CT imaging”. The imaging for the image diagnosis may be imaging accompanied by the administration of a contrast agent. The body surface is, for example, the skin of the whole body including the face, and mucous membranes such as lips and eyes. The nails are also included in the skin. As the color information, for example, a combination of an R component, a G component, and a B component may be applied, a combination of a hue, a brightness, and a saturation may be applied, or both the combinations may be applied.

Specifically, the acquisition unit 30 may extract the color information from the visible light image obtained by performing the visible light imaging on the subject H with the camera 7. In addition, the acquisition unit 30 may extract the color information in a predetermined region of the body surface of the subject H from the visible light image. This is because, even in a case where there is a change in the health state, the color of the body surface does not change uniformly, and only a part of the region changes partially or mottledly in many cases.

As the predetermined region on the body surface, it is preferable to apply a region in which the color is likely to change depending on the health state of the subject H. For example, a region including at least one of the skin of the subject H or a mucous membrane such as the lips and the eyes is suitable. That is, it is preferable that the color information is color information in at least one of the skin or the mucous membrane of the subject H. In addition, the face is particularly suitable in the whole body. That is, it is preferable that the color information is color information in the face of the subject.

In addition, the acquisition unit 30 repeatedly acquires the color information of at least a part of the body surface of the subject H over time from before the start of the CT imaging to during the CT imaging. Here, the color information that is a reference for the health state of the subject H is referred to as reference color information. For example, the acquisition unit 30 may acquire the color information of the body surface of the subject H before the start of the CT imaging as the reference color information. In addition, for example, the acquisition unit 30 may acquire the color information of the body surface of the subject H that is first acquired after the start (that is, immediately after the start) of the CT imaging, as the reference color information.

The estimation unit 32 monitors the health state of the subject H undergoing the CT imaging using the estimation model 40 for estimating the change in the color information in a case where the health state of the subject H changes. Specifically, the estimation unit 32 generates color information (hereinafter, referred to as abnormal color information) in a case where the health state of the subject H is abnormal by inputting the reference color information to the estimation model 40.

The estimation model 40 is a trained model for causing the computer to function to receive the reference color information as an input and output the abnormal color information. As the estimation model 40, for example, a machine learning model such as a convolutional neural network and a recurrent neural network can be applied.

The training data of the estimation model 40 is a combination of the color information in a case where the health state is normal and the color information in a case where the health state is abnormal. Since there is an individual difference in the color of the body surface, it is preferable to perform learning using a combination of color information in a normal state and color information in an abnormal state for the body surface of various people. Through the machine learning using such training data, the estimation model 40 is trained to output abnormal color information suitable for the input reference color information regardless of the color of the body surface of the subject H.

In addition, the estimation model 40 may output a plurality of pieces of the abnormal color information in a case where the health state of the subject H indicates a plurality of different types of abnormal states. For example, in CT imaging, it is assumed that the subject H is in poor physical condition due to the influence of the contrast agent and is in a cyanosis state due to oxygen deficiency. In this case, the estimation model 40 may output the abnormal color information in a case where the subject H is in poor physical condition due to the influence of the contrast agent and the abnormal color information in a case where the subject H is in a cyanosis state. In addition, a plurality of estimation models 40 may be generated for each type of the abnormal states.

Next, the estimation unit 32 compares the color information during the CT imaging with the abnormal color information each time the color information is acquired during the CT imaging. Then, in a case where the comparison result satisfies a predetermined condition, it is estimated that the health state of the subject His abnormal. The predetermined condition is, for example, a case where the difference between the color information and the abnormal color information during the CT imaging is equal to or less than a predetermined threshold value. The difference between the color information is obtained by, for example, a Euclidean distance.

In a case where the plurality of pieces of abnormal color information are generated for each type of abnormal state, the estimation unit 32 may estimate that the health state of the subject H is abnormal in a case where a comparison result between the color information during the CT imaging and at least one of the plurality of pieces of abnormal color information satisfies the predetermined condition. For example, in a case where the predetermined condition is satisfied for only one of the abnormal color information in a case where the subject H is in poor physical condition due to the influence of the contrast agent and the abnormal color information in a case where the subject H is in a cyanosis state, it may be estimated that the health state of the subject H is abnormal.

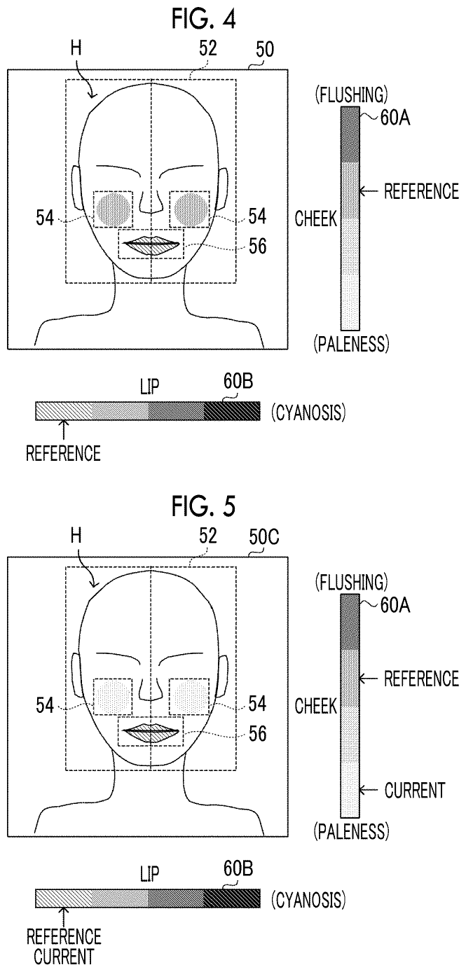

An example of the processing of the acquisition unit 30 and the estimation unit 32 will be described with reference to FIGS. 4 and 5. FIG. 4 shows a visible light image 50 acquired before the start of the CT imaging. FIG. 5 shows a visible light image 50C acquired in a case where the health state is abnormal during the CT imaging. Here, it is assumed that the CT imaging is imaging accompanied by the administration of the contrast agent, and it is possible to estimate whether or not the subject His in poor physical condition due to the influence of the contrast agent from the degree of flushing and paleness of the cheek of the subject H. In addition, it is possible to estimate whether or not the subject H is in the cyanosis state due to oxygen deficiency from the color of the lips of the subject H.

The acquisition unit 30 acquires the visible light image 50 acquired before the start of the CT imaging shown in FIG. 4. In addition, the acquisition unit 30 detects a face region 52 from the visible light image 50. Then, the acquisition unit 30 detects a check region 54 and a lip region 56 from the face region 52. As a method of detecting each region, a known image recognition technology can be appropriately applied.

Next, the acquisition unit 30 extracts color information, such as the R component, the G component, and the B component, for each of the detected cheek region 54 and lip region 56. The color information extracted from the visible light image 50 acquired before the start of the CT imaging is the reference color information for each of the cheek region 54 and the lip region 56.

The estimation unit 32 generates the abnormal color information for each of the cheek region 54 and the lip region 56 by inputting the reference color information for each of the check region 54 and the lip region 56 to the estimation model 40. As an example, FIGS. 4 and 5 show a sample 60A and a sample 60B of the abnormal color information. The sample 60A indicates the abnormal color information of the check region 54, and is estimated to be in the abnormal state in a case where the color information is changed to the flushing side or the paleness side. The sample 60B indicates the abnormal color information of the lip region 56, and is estimated to be in the abnormal state in a case where the color information is changed to the cyanosis side.

In a case where the CT imaging is started, the acquisition unit 30 acquires the visible light image over time. In addition, the acquisition unit 30 extracts color information for each of the cheek region 54 and the lip region 56, similarly to the visible light image 50. The acquisition unit 30 may extract the color information each time the visible light image is acquired (that is, in accordance with the frame rate of the camera 7), or may extract the color information at a predetermined time interval, for example, every 1 second or every 30 frames.

The estimation unit 32 compares the color information during the CT imaging with the abnormal color information and monitors whether or not the health state of the subject H is abnormal each time the color information is acquired during the CT imaging. It is assumed that the visible light image 50C shown in FIG. 5 and color information thereof are acquired during the CT imaging. In this case, since the difference between the current color information and the abnormal color information in the check region 54 is equal to or less than a predetermined threshold value, the estimation unit 32 estimates that the subject H is in the abnormal state.

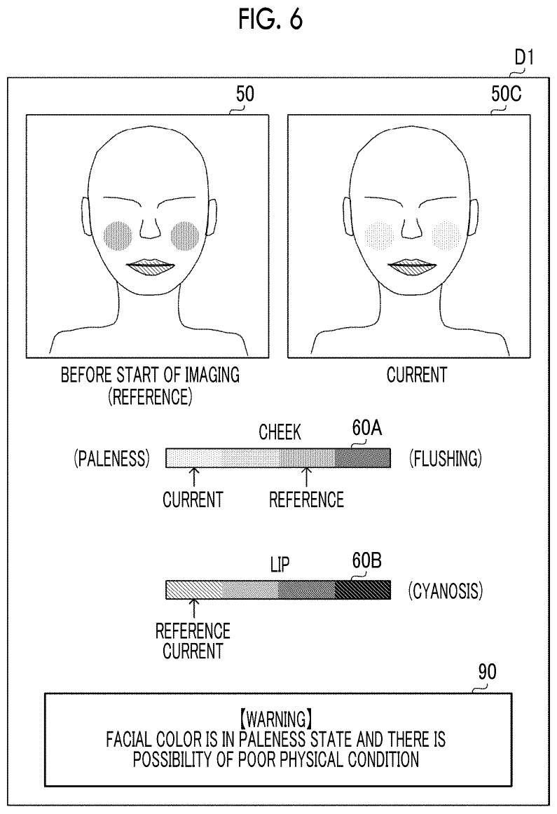

FIG. 6 shows an example of a screen D1 displayed on the display 24 by the controller 34. As shown in FIG. 6, the controller 34 may perform control to display the visible light image 50 from which the reference color information is acquired and the visible light image 50C at the current point in time. In addition, the controller 34 may perform control to display the sample 60A and the sample 60B of the abnormal color information so that the user can refer to the sample 60A and the sample 60B.

In addition, it is preferable that the controller 34 issues a warning in a case where it is estimated that the health state of the subject H is abnormal. In the example of FIG. 6, a warning message 90 indicating that the health state of the subject H is abnormal is displayed. The warning may be simple content that only informs that the state is an abnormal state, for example, without including the cause and the detailed state, or may be specific content such as a flushing state, a paleness state, and a cyanosis state. In addition, the method of warning is not limited to a sentence, and may be, for example, a method using sound, light, or the like.

In a case where it is assumed that the reference color information as the input to the estimation model 40 greatly varies for each subject H, it is preferable to prepare a plurality of the estimation models 40 according to the reference color information. That is, it is preferable that the plurality of estimation models 40 are generated according to the color information in a case where the health state of the subject H is normal. For example, in a case where learning is performed by mixing training data for different races such as Caucasoid, Negroid, and Mongoloid, there is a possibility that learning may not progress due to excessive variation or the accuracy of estimation may be reduced. In this case, by generating the estimation model 40 by dividing the training data for each race, these problems can be avoided.

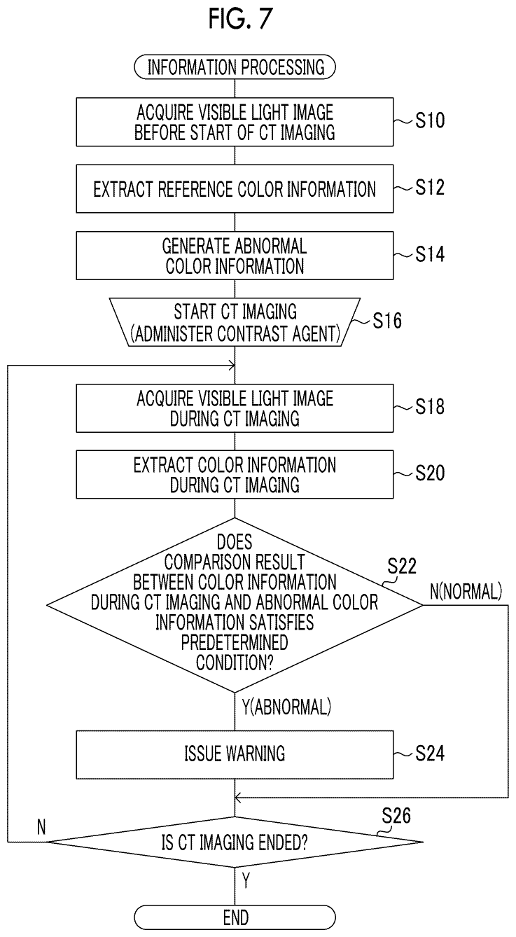

Next, an action of the information processing apparatus 10 will be described with reference to FIG. 7. In the information processing apparatus 10, as the CPU 21 executes the information processing program 27, information processing shown in FIG. 7 is executed. For example, the processing is executed in a case where the user gives an instruction to start execution.

In step S10, the acquisition unit 30 acquires a visible light image obtained by performing the visible light imaging on the subject H by the camera 7 before the start of the CT imaging. In step S12, the acquisition unit 30 extracts the color information from the visible light image acquired in step S10 as the reference color information. In step S14, the estimation unit 32 generates the abnormal color information in a case where the health state of the subject H is abnormal by inputting the reference color information extracted in step S12 to the estimation model 40.

In step S16, the user starts the CT imaging. In a case where the contrast agent is accompanied, the contrast agent is administered to the subject H. In step S18, the acquisition unit 30 acquires a visible light image obtained by performing the visible light imaging on the subject H by the camera 7 during the CT imaging. In step S20, the acquisition unit 30 extracts the color information from the visible light image acquired in step S18.

In step S22, the estimation unit 32 compares the color information during the CT imaging extracted in step S20 with the abnormal color information generated in step S14, and determines whether or not the comparison result satisfies a predetermined condition. In a case where a positive determination is made in step S22 (that is, in a case where the comparison result satisfies the predetermined condition), it is estimated that the health state of the subject H is abnormal, and the processing proceeds to step S24. In step S24, the controller 34 issues a warning that the health state of the subject H is abnormal, and the processing proceeds to step S26.

On the other hand, in a case where a negative determination is made in step S22 (that is, the comparison result does not satisfy the predetermined condition), it is estimated that the health state of the subject H is normal, and the processing proceeds to step S26. In step S26, the controller 34 determines whether or not the CT imaging is ended. In a case where a negative determination is made in step S26 (that is, in a case where the CT imaging is not ended), steps S18 to S26 are repeated. On the other hand, in a case where a positive determination is made in step S26 (that is, in a case where the CT imaging is ended), the present information processing is ended.

As described above, the information processing apparatus 10 according to the present embodiment comprises the processor. The processor acquires the color information of at least a part of the body surface of the subject H undergoing the imaging for the image diagnosis, and monitors the health state of the subject H undergoing the imaging using the estimation model 40 for estimating the change in the color information in a case where the health state of the subject H changes. Therefore, with the information processing apparatus 10 according to the present embodiment, it is possible to monitor the health state of the subject H undergoing the imaging for the image diagnosis.

In the above-described embodiment, the form in which the color information is extracted from the visible light image has been described, but the present disclosure is not limited thereto. The color information of at least a part of the body surface of the subject H may be measured by, for example, a color sensor using a photoelectric sensor.

Furthermore, at least one of functional units provided in the information processing apparatus 10 in the above-described embodiment may be provided in other devices such as a control device provided in the gantry 2 and a control device provided in the driving unit 3C.

In addition, the above embodiment has been described with reference to the CT apparatus 1, but the technology of the present disclosure can be applied to various modalities that perform the imaging for the image diagnosis other than the CT apparatus 1. Examples of the various modalities include a simple X-ray imaging apparatus, a magnetic resonance imaging (MRI) apparatus, a positron emission tomography (PET) apparatus, a mammography apparatus, an ultrasound diagnostic apparatus, an endoscope, a fundus camera, and the like.

In addition, in the above-described embodiments, for example, as hardware structures of processing units that execute various types of processing, such as the acquisition unit 30, the estimation unit 32, and the controller 34, various processors shown below can be used. As described above, in addition to the CPU that is a general-purpose processor that executes software (program) to function as various processing units, the various processors include a programmable logic device (PLD) that is a processor whose circuit configuration can be changed after manufacture, such as a field programmable gate array (FPGA), and a dedicated electric circuit that is a processor whose circuit configuration is designed for exclusive use in order to execute specific processing, such as an application-specific integrated circuit (ASIC).

One processing unit may be configured by one of the various processors or may be configured by a combination of two or more processors of the same type or different types (for example, a combination of a plurality of FPGAs or a combination of a CPU and an FPGA). A plurality of processing units may be configured by one processor.

A first example of the configuration in which the plurality of processing units are configured by one processor is a form in which one processor is configured by a combination of one or more CPUs and the software and this processor functions as the plurality of processing units, as represented by computers such as a client and a server. Second, there is a form in which, as typified by a system on chip (SoC) and the like, a processor that implements functions of an entire system including a plurality of processing units with one integrated circuit (IC) chip is used. As described above, the various processing units are configured by using one or more of the above various processors as the hardware structure.

Furthermore, as the hardware structure of these various processors, more specifically, an electric circuit (circuitry) in which circuit elements such as semiconductor elements are combined can be used.

In the above embodiment, the form in which the information processing program 27 is stored (installed) in the storage unit 22 in advance has been described, but the present disclosure is not limited thereto. The information processing program 27 may be provided in a form recorded in a recording medium such as a compact disc read only memory (CD-ROM), a digital versatile disc read only memory (DVD-ROM), and a Universal Serial Bus (USB) memory. In addition, the information processing program 27 may be downloaded from an external device via a network.

In addition, the present disclosure can also be applied to a program and a program product. Specifically, the information processing program 27 in the above-described embodiment may be provided as a program product. The program product includes products in all aspects for providing a program. For example, the program product includes a program provided through a network such as the Internet, and a computer-readable recording medium that non-transitorily stores the program.

In the technology of the present disclosure, the embodiment and the modification examples described above can be combined as appropriate. The contents described and shown above are detailed descriptions of portions related to the technology of the present disclosure and are merely an example of the technology of the present disclosure. For example, description related to the above configurations, functions, actions, and effects is description related to an example of configurations, functions, actions, and effects of the parts according to the technology of the present disclosure. As a result, it goes without saying that unnecessary parts may be deleted, new elements may be added, or replacements may be made with respect to the above-described contents and the above-shown contents within a range that does not deviate from the gist of the technology of the present disclosure.

Further, the following Supplementary Notes will be disclosed with regard to the above embodiments.

### Supplementary Note 1

An information processing apparatus comprising:
a processor,
in which the processor is configured to:
   acquire color information of at least a part of a body surface of a subject undergoing imaging for image diagnosis; and
   monitor a health state of the subject undergoing the imaging using an estimation model for estimating a change in the color information in a case where the health state of the subject changes.

### Supplementary Note 2

The information processing apparatus according to Supplementary Note 1,
in which the estimation model is a learning model that is trained in advance to receive input of reference color information that is the color information serving as a reference for the health state of the subject and output abnormal color information that is the color information in a case where the health state of the subject is abnormal, and
the processor is configured to:
   acquire, as the reference color information, the color information of the body surface of the subject before a start of the imaging;
   generate the abnormal color information by inputting the reference color information to the estimation model; and
   estimate that the health state of the subject is abnormal in a case where a comparison result between the color information during the imaging and the abnormal color information satisfies a predetermined condition.

### Supplementary Note 3

The information processing apparatus according to Supplementary Note 2,
in which the estimation model outputs a plurality of pieces of the abnormal color information in a case where the health state of the subject indicates different types of abnormal states, and
the processor is configured to:
   estimate that the health state of the subject is abnormal in a case where a comparison result between the color information during the imaging and at least one of the plurality of pieces of abnormal color information satisfies a predetermined condition.

### Supplementary Note 4

The information processing apparatus according to Supplementary Note 2 or 3,
in which the estimation model is trained by machine learning using a combination of the color information in a case where the health state is normal and the color information in a case where the health state is abnormal as training data.

### Supplementary Note 5

The information processing apparatus according to any one of Supplementary Notes 1 to 4,
in which the processor is configured to:
   issue a warning in a case where it is estimated that the health state of the subject is abnormal.

### Supplementary Note 6

The information processing apparatus according to any one of Supplementary Notes 1 to 5,
in which the processor is configured to:
   extract the color information from a visible light image obtained by performing visible light imaging on the subject.

### Supplementary Note 7

The information processing apparatus according to Supplementary Note 6,
in which the processor is configured to:
   extract the color information in a predetermined region of the body surface of the subject from the visible light image.

### Supplementary Note 8

The information processing apparatus according to any one of Supplementary Notes 1 to 7,
in which the color information is color information in at least one of a skin or a mucous membrane of the subject.

### Supplementary Note 9

The information processing apparatus according to any one of Supplementary Notes 1 to 8,
in which the color information is color information in a face of the subject.

### Supplementary Note 10

The information processing apparatus according to any one of Supplementary Notes 1 to 9,
in which the imaging is imaging accompanied by administration of a contrast agent.

### Supplementary Note 11

The information processing apparatus according to any one of Supplementary Notes 1 to 10,
in which a plurality of the estimation models are generated according to the color information in a case where the health state of the subject is normal.

### Supplementary Note 12

An information processing method executed by a computer, comprising:
acquiring color information of at least a part of a body surface of a subject undergoing imaging for image diagnosis; and
monitoring a health state of the subject undergoing the imaging using an estimation model for estimating a change in the color information in a case where the health state of the subject changes.

### Supplementary Note 13

An information processing program causing a computer to execute a process comprising:
acquiring color information of at least a part of a body surface of a subject undergoing imaging for image diagnosis; and
monitoring a health state of the subject undergoing the imaging using an estimation model for estimating a change in the color information in a case where the health state of the subject changes.

### Supplementary Note 14

A trained model for causing a computer to function as follows:
receiving input of reference color information that is color information of at least a part of a body surface of a subject on whom imaging for image diagnosis is performed and serves as a reference for a health state of the subject; and outputting abnormal color information that is the color information in a case where the health state of the subject is abnormal,
in which the trained model is trained in advance by machine learning using a combination of the color information in a case where the health state is normal and the color information in a case where the health state is abnormal as training data.

What is claimed is:

1. An information processing apparatus comprising a processor, wherein the processor is configured to:
acquire color information of at least a part of a body surface of a subject undergoing imaging for image diagnosis; and
monitor a health state of the subject undergoing the imaging using an estimation model for estimating a change in the color information in a case where the health state of the subject changes.

2. The information processing apparatus according to claim 1, wherein:
the estimation model is a learning model that is trained in advance to receive input of reference color information that is the color information serving as a reference for the health state of the subject and output abnormal color information that is the color information in a case where the health state of the subject is abnormal, and
the processor is configured to:
acquire, as the reference color information, the color information of the body surface of the subject before a start of the imaging;
generate the abnormal color information by inputting the reference color information to the estimation model; and
estimate that the health state of the subject is abnormal in a case where a comparison result between the color information during the imaging and the abnormal color information satisfies a predetermined condition.

3. The information processing apparatus according to claim 2, wherein:
the estimation model outputs a plurality of pieces of the abnormal color information in a case where the health state of the subject indicates different types of abnormal states, and
the processor is configured to:
estimate that the health state of the subject is abnormal in a case where a comparison result between the color information during the imaging and at least one of the plurality of pieces of abnormal color information satisfies a predetermined condition.

4. The information processing apparatus according to claim 2, wherein the estimation model is trained by machine learning using a combination of the color information in a case where the health state is normal and the color information in a case where the health state is abnormal as training data.

5. The information processing apparatus according to claim 1, wherein the processor is configured to issue a warning in a case where it is estimated that the health state of the subject is abnormal.

6. The information processing apparatus according to claim 1, wherein the processor is configured to extract the color information from a visible light image obtained by performing visible light imaging on the subject.

7. The information processing apparatus according to claim 6, wherein the processor is configured to extract the color information in a predetermined region of the body surface of the subject from the visible light image.

8. The information processing apparatus according to claim 1, wherein the color information is color information in at least one of a skin or a mucous membrane of the subject.

9. The information processing apparatus according to claim 1, wherein the color information is color information in a face of the subject.

10. The information processing apparatus according to claim 1, wherein the imaging is imaging accompanied by administration of a contrast agent.

11. The information processing apparatus according to claim 1, wherein a plurality of the estimation models are generated according to the color information in a case where the health state of the subject is normal.

12. An information processing method executed by a computer, comprising:
acquiring color information of at least a part of a body surface of a subject undergoing imaging for image diagnosis; and
monitoring a health state of the subject undergoing the imaging using an estimation model for estimating a change in the color information in a case where the health state of the subject changes.

13. A non-transitory computer-readable storage medium storing an information processing program causing a computer to execute a process comprising:
acquiring color information of at least a part of a body surface of a subject undergoing imaging for image diagnosis; and
monitoring a health state of the subject undergoing the imaging using an estimation model for estimating a change in the color information in a case where the health state of the subject changes.

14. A non-transitory computer-readable storage medium storing a trained model for causing a computer to function as follows:
receiving input of reference color information that is color information of at least a part of a body surface of a subject on whom imaging for image diagnosis is performed and serves as a reference for a health state of the subject; and outputting abnormal color information that is the color information in a case where the health state of the subject is abnormal,
wherein the trained model is trained in advance by machine learning using a combination of the color information in a case where the health state is normal and the color information in a case where the health state is abnormal as training data.
